(19) United States
(12) Patent Application Publication
(10) Pub. No.: US 2005/0163736 A1
(43) Pub. Date: Jul. 28, 2005

(54) Dual phase stick

(76) Inventors: Heng Cai, Yardley, PA (US); Aixing Fan, Bridgewater, NJ (US)

Correspondence Address: Colgate-Palmolive Company, 909 River Road, P.O. Box 1343, Piscataway, NJ, 08855-1343, US

(21) Appl. No.: 10/766,229

(22) Filed: Jan. 27, 2004

(51) Int. Cl. A61K 7/32

(52) U.S. Cl. 424/65

(57) ABSTRACT

A dual phase stick antiperspirant and/or deodorant product comprising a non-gelled opaque inner core as 20-70 weight % of the total composition and a clear outer shell as 30-80 weight % of the total composition wherein: (a) based on the weight of the inner core as 100%, the composition of the inner core comprises: (i) 20-70 weight % of an antiperspirant active powder, provided that the total amount of antiperspirant active in the total product does not exceed 25 weight %; (ii) 0.1-20 weight % of a filler; (iii) 2-20 weight % of a binder; (iv) 1-20 weight % of an emollient; (b) based on the weight of the outer shell as 100%, the clear outer shell is formed around the inner core and comprises: (i) 7-15 weight % of a selected siliconized polyamide of Formula IIIA as described; (ii) 8-80 weight % of a silicone component comprising at least 8-30 weight % of a volatile silicone; (iii) 10-70 weight % of a non-silicone organic material; (iv) optionally a co-gellant selected from the described group; wherein the outer gelled shell comprises the polyamide in combination with the silicone and non-silicone organic materials so that the ratio of the non-silicone organic materials to the organosilicone materials is in the range of 10:1-0.01:1.

This invention relates to a dual phase antiperspirant and/or deodorant stick having an opaque inner core comprising an active ingredient in a compressed powder matrix and a transparent outer shell comprising a clear anhydrous gel solid provide smooth skin feel.

### FIELD OF THE INVENTION

The use of polyamides in cosmetic compositions, especially antiperspirants and/or deodorants, has certain advantages, especially with regard to clarity and structural integrity. The use of polyamides has allowed for the creation of new products and this invention takes the formulation in yet another direction by creating a dual phase product.

### BACKGROUND OF THE INVENTION

The present invention is directed to improved cosmetic compositions formed with a specific group of polyamide gelling agents previously described in (1) U.S. Pat. No. 6,051,216 (WO 99/06473); (2) U.S. Patent Publication 2002/0051758; (3) U.S. Provisional patent application No. 60/229,444, filed on Aug. 31, 2000; (4) a second application based on (3) as a continuation-in-part case, referenced as U.S. Ser. No. 09/922,091, all of which are incorporated by reference herein in their entirety.

U.S. Pat. No. 6,451,295 (PCT Publication WO 02/178700), co-owned by one of the same owners as the present application, describes clear antiperspirants and deodorants made with siloxane-based polyamides.

PCT Publication WO 02/17871, owned by the same owner as the present application, describes antiperspirants and deodorants with low white residue on skin and fabric. The compositions comprise selected siliconized polyamides and a co-gellant.

A number of references, particularly several references previously listed above, have previously disclosed and described various types of polyamide compounds suitable for use in cosmetic products. The polyamides may be used to form stick compositions by gelling at least one silicone fluid (for example, silicone liquids such as silicone oils) which is thickened using a siliconized polyamide as a gelling agent. For these references, a single form is described which incorporates the antiperspirant active in the total composition.

U.S. Pat. No. 5,243,010 to Choi, et al., discloses aromatic polyamide resins having pendant silyl groups.

U.S. Pat. No. 5,272,241 to Lucarelli, et al., discloses organofunctional siloxanes useful in both the personal care and plastics industries, the siloxanes being amino acid functionalized silicones.

U.S. Pat. No. 5,919,441, assigned to The Mennen Company describes in general the use of polyamides as gelling agents for cosmetic compositions.

Co-gellants may also be used with the polyamide gelling agent as described in U.S. Patent Publication 2002/0051758. Such gellant systems include those made with an n-acyl amino acid such as N-lauroyl-glutamic acid derivatives. Examples of such gelling systems include those described in U.S. Pat. Nos. 3,969,087; 5,429,816; 5,733,534; 5,776,494; 5,591,424; 5,840,287; 5,843,407; 5,846,520; 5,849,276; 5,965,113; 6,190,673; and 6,241,976.

Other references include U.S. Pat. No. 5,120,531 to Wells, et al discloses rinse-off hair conditioner and styling compositions providing a gel-network thickened vehicle for the styling polymer and solvent. This patent discloses various siloxanes as the conditioning agent including polydiorganosiloxanes having quaternary ammonium-substituted groups attached to the silicon, and polydiorganosiloxanes having silicone-bonded substituents which are amino-substituted hydrocarbon groups.

U.S. Pat. No. 5,500,209 discloses a gel or stick which includes active deodorant and/or antiperspirant ingredients, a polyamide gelling agent, and a solvent for the polyamide gelling agent, in which the gel or stick composition can be clear or translucent. This patent discloses that the polyamide gelling agent is soluble in a cosmetically acceptable solvent at elevated temperatures, and solidifies (gels) upon cooling; acceptable solvents are disclosed as including various alcohols, including various glycols. While the polyamide-containing stick or gel disclosed in the aforementioned patent contains desirable properties in connection with stability of the composition, (particularly in the presence of acidic antiperspirant active materials, and in providing clear or translucent gel or stick compositions) such formulas may result in tackiness and stickiness both upon and after application to the skin.

Addressing this problem of tackiness and stickiness in connection with cosmetic compositions utilizing a polyamide gelling agent, U.S. Pat. No. 5,603,925, the contents of which are incorporated herein by reference in their entirety, discloses the use of a specific solvent system for a solid composition containing an antiperspirant active material and a polyamide gelling agent. This solvent system is glycol-free and contains a non-ionic surfactant and a polar solvent. Water is the polar solvent, and the non-ionic surfactant acts as a dispersing medium for the antiperspirant active material, in which sufficient water is used to give a clear or translucent solution/emulsion of the antiperspirant active material.

Antiperspirant products are well known in the art. Antiperspirant products have appeared in the marketplace in various dosage forms, such as sticks, gels, roll-ons, aerosols and creams. Generally, these dosage forms include a solution of the active ingredient in a solvent, a suspension of the active ingredient in a non-solvent, or a multi-phase dispersion or emulsion in which a solution of the active ingredient is dispersed in some continuous phase or in which the solubilized active ingredient constitutes a continuous phase. Of the above-referred-to dosage forms, the stick form can be distinguished from other forms on the basis that in a stick the formulated product can maintain its shape for extended time periods outside the package (allowing for some shrinkage due to solvent evaporation).

A typical technique to reduce the tackiness of, for example, antiperspirant formulations is the incorporation of one or more cyclomethicones (penta- or hexa-cyclodimethyl-siloxanes or mixtures thereof). These cyclomethicones are very low-viscosity silicone liquids that provide excellent lubricity but do not leave stains on the skin and/or clothing. More than 50% by weight of cyclomethicone has been incorporated into solid stick antiperspirant formulations, for example, using a wax solidifying agent. However, cyclomethicone is a nonsolvent for the dimer based polyamides described as gelling agents in U.S. Pat. No. 5,500,209. Moreover, only limited quantities of the cyclomethicone can be incorporated in solid compositions gelled using such polyamide gelling agent, without destroying the clarity of the gelled composition. Beyond that point, the gelled composition becomes cloudy because of either excessive crystallization of the polyamide or immiscibility of the cyclomethicone in the mixture.

Notwithstanding the foregoing, there is still a need for improved siloxane-based polyamide gelling agents and cosmetic compositions made therefrom, especially when stick products made with such polyamides are capable of forming cosmetic products having improved clarity and physical integrity. The present invention provides a novel way of providing a product with a clear outer shell and an opaque core. This enables the structural integrity of the outer transparent core to be improved.

Thus, it is an object of the present invention to provide an improved cosmetic composition as a dual phase antiperspirant and/or deodorant stick, comprising a selected siloxane-based polyamide as a gelling agent which cosmetic composition is capable of exhibiting improved aesthetics such as clarity and which preferably leaves low to no visible white residue upon application and after drying It is also an overall object of the present invention to provide a dual phase antiperspirant and/or deodorant stick comprising an outer phase solidified (also referred to as being gelled) with a selected siloxane-based polyamide and exhibiting a dry feel and minimum tack. A further object of the invention is to provide a product with a pleasing visual impact with better efficacy.

### SUMMARY OF THE INVENTION

The composition of the invention comprises:
(a) a non-gelled inner core made of compressed powder wherein the core comprises an antiperspirant active powder, filler, binder, an emollient and a surfactant; and
(b) a clear outer solid phase (also referred to as outer shell or gelled outer shell) as an anhydrous gel comprising a polyamide gelled composition comprising a selected polyamide as described below, one or more silicone fluids, one or more emollients and, optionally, a co-gellant;
   wherein the ratio of the core to the shell based on weight % is 20-70:30-80.

### DETAILED DESCRIPTION OF THE INVENTION

The invention comprises a dual phase system with an inner core and an outer shell positioned around the inner core. The inner core is opaque and the outer shell is clear.

The inner core can be made in a variety of shapes and dimensions including oval, round square and rectangular. The outer shell may either be positioned continuously around the inner core or may have breaks in the continuity of the outer shell around the inner core (for example, a horseshoe shape for the outer shell around the inner core). The inner core comprises about 20-70 weight % of the total product. Based on the weight of the inner core as 100%, the composition of the inner core comprises:
(a) 20-70 weight % (particularly 25-50 weight %) of an antiperspirant active powder, provided that the total amount of antiperspirant active in the total product does not exceed 25 weight %;
(b) 0.1-20 weight % of a filler;
(c) 2-20 weight % of a binder;
(d) 1-20 weight % (particularly 1-10 weight %) of an emollient;
(e) optionally 0-3 weight % of a surfactant (for example, 0.5-3%); and
(f) optionally 0-3 weight % of one or more members selected from the group consisting of coloring agents, and fragrances.

The antiperspirant active can be selected from the powder actives normally used in such products. These include, by way of example (and not of a limiting nature), aluminum chlorohydrate, aluminum chloride, aluminum sesquichlorohydrate, zirconyl hydroxychloride, aluminum-zirconium glycine complex (for example, aluminum zirconium trichlorohydrex gly, aluminum zirconium pentachlorohydrex gly, aluminum zirconium tetrachlorohydrex gly and aluminum zirconium octochlorohydrex gly), aluminum chlorohydrex PG, aluminum chlorohydrex PEG, aluminum dichlorohydrex PG, and aluminum dichlorohydrex PEG. The aluminum-containing materials can be commonly referred to as antiperspirant active aluminum salts. Generally, Category I active antiperspirant ingredients listed in the Food and Drug Administration's Monograph on antiperspirant drugs for over-the-counter human use can be used. In addition, any new drug, not listed in the Monograph, such as aluminum nitratohydrate and its combination with zirconyl hydroxychlorides and nitrates, or aluminum-stannous chlorohydrates, can be incorporated as an antiperspirant active ingredient in antiperspirant compositions according to the present invention.

Particular types of antiperspirant actives include aluminum zirconium trichlorohydrex and aluminum zirconium tetrachlorohydrex either with or without glycine. A particular antiperspirant active is aluminum trichlorohydrex gly such as AZZ-902 SUF (from Reheis Inc., Berkley Heights, N.J.) which has 95% of the particles less than 10 microns in size and AA ZG 7167 and AA ZG 7168 (from Summit Research Labs, Huguenot, N.Y.) which also has 95% of the particles less than 10 microns in size.

Another particular type of antiperspirant salt of interest is the group that has a low metal to chloride ratio such as in the range of 0.9-1.2:1. Examples of such salts are described in U.S. Pat. No. 6,375,937.

Antiperspirant actives can be incorporated into the core composition according to the present invention in amounts in the range of 0.1-25 weight %, 5-25%, and preferably 15-25 weight % based on the total weight of the total composition. The amount used will depend on the formulation of the composition. For example, at amounts in the lower end of the broader range (for example, 0.1-10%), the antiperspirant active material will not substantially reduce the flow of perspiration, but will reduce malodor, for example, by acting as an antimicrobial material. It should also be noted that the Monograph establishes useful ranges for the total product. The antiperspirant is preferably placed only in the inner core, but a portion of the active can be included in the outer shell. The inclusion of the antiperspirant active in the outer shell, however, makes it harder to maintain structural integrity. For example, the antiperspirant active material is desirably included as particulate matter suspended in the core of the present invention in amounts as described above with no antiperspirant present in the outer shell.

The core part of the composition also includes inert filler material selected from those useful in cosmetic formulations. Acceptable fillers include those selected from the group consisting of inorganic oxides (for example, aluminum oxide, titanium dioxide, zinc oxide), silicates (for example, silica, various types of fumed silica, magnesium silicates, talc, talcum powder, clay (especially inorganic clays such as kaolin or bentonite), zinc stearate, cornstarch, polyethylene (especially in the size range of 10-50 microns), Nylon-12 and mixtures of these inert particulate materials. In one particular version of the product, the inert filler, in particulate form, has physical properties (for example, shape, etc.) that are similar to those of the antiperspirant active material (for example, antiperspirant active metal salt).

Binders may be selected from the group consisting of acrylates copolymers (such as acrylates/polyvinylpyrrolidone copolymer and acrylates/vinyl acrylate copolymer); acrylic acid and its copolymers (such as acrylic acid/acrylonitrogens copolymer); polyvinylpyrrolidone; copolymers of PVP (such as polyvinylpyrrolidone/vinyl acrylate copolymer); agar; algin; C10-200 alkyl alcohols (with one particular group being C 10-30 alkyl alcohols, and another particular group consisting of the group containing behenyl alcohol, lanolin alcohol, and polyvinyl alcohols (“PVA”) such as PVA's having a total number of carbons of 50-200); waxes having a melting point in the range of 40-100 degrees C. (such as natural synthetic or modified waxes selected from the group consisting of beeswax, candelilla wax, synthetic wax, and silicone wax); gums (such as cellulose gum and xanthan gum); celluloses (such as hydroxyethylcellulose and hydroxypropylcellulose); gelatin; starches (such as glyceryl starch, cornstarch; and sodium polyacrylate starch); lanolins, guars (such as hydroxypropyl guar and carboxymethyl hydroxypropyl guar); isopropyl/isostearyl esters, polyethylene glycols (such as PEG-32, PEG-350, and PEG-90M); other agents such as dibenzylidene sorbitol, 12-hydroxystearic acid, and N-acyl amino acids; and dimethicone copolyol.

One particular set of stick products are those wherein the binder is selected from the group consisting of (a) acrylates copolymers selected from the group consisting of acrylates/polyvinylpyrrolidone copolymers and acrylates/vinyl acrylate copolymers; (b) acrylic acid and its copolymers selected from the group consisting of acrylic acid/acrylonitrogens copolymers; (c) polyvinylpyrrolidone; (d) copolymers of polyvinylpyrrolidone selected from the group consisting of polyvinylpyrrolidone/vinyl acrylate copolymers); (e) agar; (f) algin; (g) C10-200 alkyl alcohols; (h) waxes having a melting point in the range of 40-100 degrees C.; (i) gums selected from the group consisting of cellulose gums and xanthan gums; (j) celluloses selected from the group consisting of hydroxyethylcellulose and hydroxypropylcellulose; (k) gelatin; (l) starches selected from the group consisting of glyceryl starch, cornstarch; (m) lanolins; (n) guars selected from the group consisting of hydroxypropyl guar and carboxymethyl hydroxypropyl guar); (o) isopropyl/isostearyl esters; (p) polyethylene glycols selected from the group consisting of PEG-32, PEG-350, and PEG-90M; (q) dimethicone copolyol; and (r) mixtures of any of the foregoing.

Emollients may also be included in the inner core composition. Illustratively these include (but are not limited to):

(a) ethoxylated/propoxylated surfactants having 4-48 carbon atoms and 3-15 EO or PO units, such as PPG-14 butyl ether (for example, Fluid AP by Union Carbide Corp.), PEG-8-distearate;
(b) oils (such as mineral oil, avocado oil, castor oil, corn oil, jojoba oil, safflower oil, sesame oil, soybean oil, and squalene);
(c) hydrogenated oils (such as hydrogenated vegetable oil, hydrogenated jojoba oil, and hydrogenated castor oil);
(d) non-silicone organics not otherwise included in (a) or (b) or (c) and which are described below for the outer shell; and
(e) volatile silicones as described below for the outer shell (note that one embodiment of the invention, however, is a product which is free of any silicones in the inner core).

In one particular embodiment, the emollient is selected from (a), (b), (c) and mixtures thereof.

Another embodiment provides a stick product wherein the emollient is selected from the group consisting of:
(a) ethoxylated/propoxylated surfactants having 4-48 carbon atoms and 3-15 EO or PO units;
(b) oils selected from the group consisting of mineral oil, avocado oil, castor oil, corn oil, jojoba oil, safflower oil, sesame oil, soybean oil, and squalene;
(c) hydrogenated oils selected from the group consisting of hydrogenated vegetable oil, hydrogenated jojoba oil, and hydrogenated castor oil;
(d) non-silicone organics not otherwise included in (a) or (b) or (c) and which are described elsewhere in the application for the outer shell as non-silicone organics or emollients for the outer shell;
(e) volatile silicones; and
(f) mixtures of any of the foregoing.

Surfactants are an optional ingredient which may be added to the inner core in amounts up to 3 weight % based on the weight of the inner core, for example as a minimum of 0.5 weight %. Such surfactants may be selected from the group consisting of:
(a) alkanolamides (such as N-alkyl pyrrolidone),
(b) ethoxylated amides (for example, the polyethylene glycol amide of tallow acid that conforms generally to the formula RC(O)—NH—(CH₂CH₂O)ₙH where RCO-represents the fatty acids derived from tallow and n has an average value of 50 (also called PEG-50 tallow amide));
(c) amine oxides (for example, cocamidopropylamine oxide);
(d) ethoxylated fatty acids (for example, the polyethylene glycol diester of stearic acid that conforms generally to the formula CH₃(CH₂)₁₆C(O)—(OCH₂CH₂)ₙO—C(O)(CH₂)₁₆CH₃ (also called PEG-8 distearate));
(e) ethoxylated glycerides (for example, a polyethylene glycol derivative of Castor Oil with an average of 4 moles of ethylene oxide (also called PEG-4 castor oil));
(f) glycol esters (for example, propylene glycol ricinoleate);
(g) monoglycerides (for example, glycerol myristate);
(h) polyglyceryl esters (for example, polyglyceryl-4 oleyl ether);
(i) polyhydric alcohol esters and ethers (for example, sucrose distearate);
(j)sorbitan/sorbitan esters (for example, sorbitan sesquiisostearate);
(k) triesters of phosphoric acid (for example, trioleth-8 phosphate (a material which is predominantly the triester of phosphoric acid and ethoxylated oleyl alcohol with an average of 8 moles of ethylene oxide));
(l) ethoxylated lanolin (for example, a polyethylene glycol derivative of Lanolin with an average of 20 moles of ethylene oxide (also called PEG-20 lanolin));
(m) propoxylated polyoxyethylene ethers (for example, the polyoxypropylene, polyoxyethylene ether of cetyl alcohol that conforms generally to the formula CH₃(CH₂)₁₄CH₂(OCH(CH₃)CH₂)ₓ(OCH₂CH₂)_{y}OH
   where x has an average value of 5 and y has an average value of 20 (also called PPG-5 ceteth-20)); and
(n) alkylpolyglycosides (for example, lauryl glucose).

Other optional ingredients for the core include minor amounts (up to 5 weight %) of minerals (such as calcium citrate, magnesium citrate, di-potassium aspartate, zinc picolinate, manganese aspartate, iron glycinate, and copper glycinate).

The second phase of the dual phase composition is a clear cosmetic composition which forms an outer shell or gelled shell around the core which shell is formed by combining (based on the weight of the gelled shell):
(a) 7-15 weight % of the selected polyamide as described below (especially at least 8 weight % of a polyamide of Formula IIIA with DP=15);
(b) 8-80 weight % of a silicone component comprising at least 8-30 weight % of a volatile silicone and optionally 0-30 weight % of a non-volatile silicone;
(c) 10-70 weight % of a non-silicone organic material;
(d) optionally a co-gellant selected from the group consisting of: 0-1.7% dibenzylidene sorbitol (only in anhydrous systems); 0-0.5% 12-hydroxy stearic acid; 0-0.5% amine stearate; 0-0.5% N,N′-hexamethylenebis-(10-undecenamide; 0-5.0% silicone elastomer (for example, DC-9040 form Dow Corning Corp. or KSG-15 from Shin-Etsu); and 0-1% N-lauroyl glutamic acid amide (for example, GP-1 from Ajinomoto);
(e) optionally 0-5 weight % of a glycol component; and
(f) 0-3 weight % of a surfactant (particularly polysorbate 20 or polysorbate 80) with a particular minimum being 0.5 weight %; wherein the outer gelled shell comprises the polyamide in combination with the silicone and non-silicone organic materials so that the ratio of the non-silicone organic materials to the organosilicone materials is in the range of 10:1-0.01:1.

The solvent for the outer shell comprises the silicone (volatile silicone and optionally a non-volatile silicone) and the non-silicone organic material. The solvent is gelled by the thickening agent (which thickening agent will include at least one polyamide as described above and, optionally, a co-gellant) is included in the composition in an amount sufficient such that the thickening agent can be dissolved therein and gelled therefrom, and includes a silicone fluid (for example, a silicone oil, such as cyclomethicone and/or dimethicone). Thus, the thickening agent can be dissolved in the solvent and gelled therefrom, for example, upon cooling the composition during manufacture thereof.

The silicone fluid portion is in the range of 5-95 percent by weight, more particularly 10-80 percent by weight, even more particularly 20-50 percent by weight of the outer gelled shell.

The outer shell includes silicone fluids. These fluids can be volatile or non-volatile and include linear siloxanes known as dimethicones, linear siloxanes containing an aromatic substitution such as phenyl trimethicone and the various cyclic siloxanes having from 5-6 siloxane units in a ring optionally substituted by C1-C6 alkyl or phenyl, particularly cyclic dimethyl siloxanes such as cyclomethicones. Mixtures of such silicone fluids may also be used. Suitable volatile silicone liquids are described in U.S. Pat. No. 5,102,656 to Kasat, referenced above. Examples of other known silicone fluids for use in cosmetic compositions are disclosed in U.S. Pat. No. 4,853,214 to Orr, referenced above and are suitable for use in this invention. Other particular examples include linear volatile silicone fluids, for example, silicone liquids conventionally used in cosmetic compositions. One particular group is illustratively (and not of a limiting nature), phenyl trimethicone, cyclomethicones and/or dimethicones, and silanols such as those described in U.S. Pat. No. 5,871,720, incorporated by reference herein to the extent these compounds are described.

Preferably, the silicone fluid includes cyclomethicones. The cyclomethicone used (that is, ring size of the cyclomethicone) has an effect on the hardness of the gels formed. That is, cyclomethicone having five siloxane units produces a softer gel than that produced utilizing a material with 6 siloxane units. As the ring size of the cyclomethicone increases, the rigidity of the gel system formed increases. As described above, particular examples of suitable cyclomethicones include those having rings of 5-6 siloxane units, especially “D5”.

The outer shell may include from 0-25% (preferably 10-20%) of an organosilicone that is selected from Formula IA (or mixtures thereof): 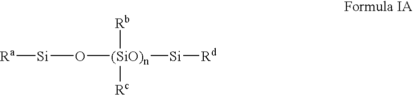
wherein each of R^{a}, R^{b}, R^{c}, and R^{d} may be the same or different and are each independently selected from the group consisting of hydrogen, C1-C15 alkyl, phenyl, and C1-C15 alkyl itself containing a member selected from the group consisting of —OH, —COOH, —NH₃—CO(O)—, and n is a number in the range of 5-500. A particular set of examples of Formula IA include those wherein each of R^{a}, R^{b}, R^{c}, and R^{d} may be the same or different and are each independently selected from the group consisting of hydrogen, C1-C15 alkyl (for example, methyl, ethyl, propyl, isopropyl), phenyl, and C1-C15 alkyl itself containing a member selected from the group consisting of, —OH, —COOH, —NH₃, —CO(O)—, and n is a number in the range of 5-500. Examples of compositions of Formula IA include phenyltrimethicone, caprylyl methicone, and phenethyl dimethicone.

Suitable functionalized silicone fluids are hydroxy functional fluids with the general structure of Formula V:
(R¹—Si—O_{3/2})ₐ—(R²₂—Si—O_{2/2})_{b}—((HO)R³₂—Si—O_{1/2})_{c} Formula V
where each of R¹, R², and R³, may be alike or different and are each independently selected from the group consisting of C1-C4 straight chain alkyls (especially methyl);
a is a number in the range of 0-10, with particular values of “a” being 0 for linear compounds and 1-10 for branched compounds (for example 6-8);
b is a number in the range of 0-10,000, with particular values of “b” being 4-6000;
c is a number in the range of 1-10, with particular values of “c” being 2 when the compound is linear and at least 3 when there is branching; provided that a and b cannot both equal zero at the same time. It is to be recognized that a, b, and c are average values (including whole numbers and fractions) and mixtures of compounds with various values for a, b, c, R¹, R², and R³ may also be used.

Examples of compounds of Formula V include:
(a) linear polydimethylsiloxanediols where a=0, b=4-6,000 (for example, an average value of 4, 40 or 6,000);
(b) linear polydimethylsiloxanediols where a=0, b=4-1,000 and c=2;
(c) multifunctional branched siloxanes where a=1-2, b=0-1,000, and c=3-4;
(d) linear polydimethylsiloxanediols where a=0, b=40 and c=2;
(e) multifunctional branched siloxanes where a=1, b=16, and c=3;
(f) multifunctional branched siloxanes where a=1-2, b=10-1,000, and c=3-4;
(g) mixtures of the particular compounds described in parts (a)-(f), for example, mixtures wherein the average structure of the mixture is described by a=0.1, b=4-6000, and c=2-7; and
(h) two component mixtures of the particular compounds described in parts (a)-(f) wherein one component is 0.1-99.9% of the composition and the other component is the remainder to 100%.
   For each of the groups listed as (a)-(f) above, particular examples of the compounds are when each of the R groups is selected to be methyl. Also, for any of the groups (a)-(g), additional silicone fluids such as dimethicone may be added, for example in amounts of 0.1-90% functionalized silicone and 10-99.9% silicone fluid or fluids.

One particular group of compounds of Formula V are linear silanols of Formula VA, especially when b=40:
HO—(R³)₂ Si—O—((R²)₂ Si—O)_{b}—Si—(R³)₂ OH Formula VA

Some of the compounds of Formula V may be purchased commercially. For methods of making other compounds of this invention descriptions of suitable methods may be found in the literature for example, U.S. Pat. No. 5,302,382 to Dow Corning; U.S. Pat. No. 3,441,537 to Stauffer Chemical Company; and Noll, W., Chemistry and Technology of Silicones, (Academic Press, Inc. Orlando, Fla. 1968) especially at pages 190-196 and 239-245, all of which are incorporated herein by reference to the extent they describe how to make these compounds.

While the hydroxy functionalized silicones described above are preferably selected to have a viscosity that does not require additional silicone materials (for example, having a viscosity in the range of up to 60,000 centistoke (cst), it is possible to use compositions which are a blend of hydroxy functionalized silicones having higher viscosities such as those having a high viscosity (>500,000 centistoke) dimethiconol in dimethicone where the dimethicone has a viscosity in the range of 5-350 centistoke (for example, DOW CORNING® 1403 Fluid).

For high viscosity functionalized silicones (for example, the silicone gums), and for the purpose of facilitating its handling and processing, these materials are generally provided as blends with another volatile or non-volatile low viscosity silicone such as CYCLOMETHICONE, or a non-volatile linear silicone fluid having a viscosity of about 5 to 350 centistoke. Such dimethyl silicone polymers terminated with hydroxyl groups have been assigned the INCI name “DIMETHICONOL” by The Cosmetics, Toiletries and Fragrance Association, Inc., Washington, D.C. (CTFA). Blends of such silicone gums with a volatile low viscosity cyclic silicone have been assigned the INCI name “CYCLOMETHICONE (and) DIMETHICONOL” by the CTFA. Other blends of such silicone gums with a non-volatile low viscosity linear silicone have been assigned the INCI name “DIMETHICONE (and) DIMETHICONOL” by the CTFA. The DIMETHICONOL content of such blends is typically in the range of about 12 to 14 percent by weight, and the blend viscosity may range from 500 to about 20,000 centistoke, generally in the range of about 4,000 to 5,000 centistoke. DIMETHICONE concentrations in the range of 10-48% are known or may be made from other concentrations.

Other volatile low viscosity methylsilicone fluids are described in U.S. Pat. No. 5,302,382 to Kasprzak, incorporated by reference herein. Examples of methylsilicone fluids having viscosities of less than about one hundred centistoke measured at twenty-five degrees Centigrade, preferably less than about two centistokes and also methylsilicone fluids having a viscosity in the range of 1-350 centistoke are disclosed.

One group of methylsilicone fluids is volatile low viscosity methylsilicone fluid containing dimethylsiloxane units and, optionally, trimethylsiloxane units.

Representative compounds are cyclopolysiloxanes of the formula [(CH₃)₂SiO]_{X}, and linear short chain siloxane compounds of the formula (CH₃)₃SiO[(CH₃)₂SiO]_{y}Si(CH₃)₃, in which x is an integer having a value of from three to ten, (especially 4-6) and y is an integer having a value of from zero to about four. The cyclopolysiloxanes have been assigned the INCI name “CYCLOMETHICONE” by The Cosmetics, Toiletries and Fragrance Association, Inc., Washington, D.C. (CTFA).

The silicone fluid phase can also, optionally, include other silicone materials even when the purpose is for reasons other than viscosity modification. Particular silicone fluids are selected so that a stable emulsion can be formed when the two phases are combined and mixed. Such materials can include, for example, other silicone fluids such as polydimethylsiloxanes, polydiethylsiloxanes, and polymethylethylsiloxanes, having a viscosity in excess of 350 centistoke and up to 2,500,000 centistoke, preferably, 350-10,000 centistoke. Further examples include cetyl dimethicone copolyol, dimethicone copolyol (such as DOW CORNING® 2501, Q2-5220 and 5324 products) (these dimethicone copolyols may also function as binders) a mixture of cyclomethicone and dimethiconol (such as DOW CORNING® 1401 product); a mixture of dimethicone and dimethiconol (such as DOW CORNING® 1403 product); cetyl dimethicone (DOW CORNING® 2502 product); and stearyl dimethicone (DOW CORNING® 2503 product).

The non-silicone organic material (which can also be considered as emollients) can be selected from the group consisting of:
(a) esters (for example, isopropyl myristate and C12-15 alkyl lactate);
(b) guerbet alcohols having 8-30 carbons, particularly 12-22 carbons (for example, isolauryl alcohol, isocetyl alcohol, isostearyl alcohol);
(c) fatty alcohols (for example, stearyl alcohol, myristyl alcohol, oleyl alcohol, isocetyl alcohol);
(d) ethoxylated and propoxylated alcohols (for example, the polyethylene glycol ether of lauryl alcohol that conforms to the formula CH₃(CH₂)₁₀CH₂(OCH₂CH₂)_{b} OH where b has an average value of 4 (also called laureth −4); PPG-14 butyl ether, where the “PPG-14” portion is the polymer of propylene oxide that conforms generally to the formula H(OCH₂C(CH₃)H)_{c}OH, where c has an average value of 14; PPG-3 myristyl ether which is the polypropylene glycol ether of myristyl alcohol that conforms to the formula CH₃(CH₂)₁₂CH₂(OCH(CH₃)CH₂)_{d}OH where d has an average value of 3; PPG-10 cetyl ether which conforms to the formula CH₃(CH₂)₁₄CH₂(OCH(CH₃)CH₂)ₑOH, where e has an average value of 10;
(e) dioctyl ether;
(f) octylmethoxycinnamate and octyl salicylate;
(g) C12-C18 alkyl benzoate (especially C12-C15 alkyl benzoate) and benzoate ester derivatives thereof (for example, isostearyl benzoate and octyl dodecyl benzoate);
(h) isoparaffins having a distillation temperature in the range of 178-207 degrees C.;
(i) dioctyl carbonate; and
(j) paraffins having a distillation temperature in the range of 372-426 degrees C. Mixtures of such organic materials can also be used, provided that the clarity of the gelled shell is maintained.

One particular stick comprises a non-silicone organic material (which can also be considered as an emollient) which is selected from the group consisting of:
(a) esters selected from the group consisting of isopropyl myristate and C12-15 alkyl lactate;
(b) guerbet alcohols having 8-30 carbons;
(c) fatty alcohols selected from the group consisting of, stearyl alcohol, myristyl alcohol, oleyl alcohol, and isocetyl alcohol;
(d) ethoxylated and propoxylated alcohols selected from the group consisting of polyethylene glycol ether of lauryl alcohol that conforms to the formula CH₃(CH₂)₁₀CH₂(OCH₂CH₂)_{b} OH where b has an average value of 4; PPG-14 butyl ether, where the PPG-14 portion is a polymer of propylene oxide that conforms generally to the formula H(OCH₂C(CH₃)H)_{c}OH, where c has an average value of 14; PPG-3 myristyl ether which is the polypropylene glycol ether of myristyl alcohol that conforms to the formula CH₃(CH₂)₁₂CH₂(OCH(CH₃)CH₂)_{d}OH where d has an average value of 3; and PPG-10 cetyl ether which conforms to the formula CH₃(CH₂)₁₄CH₂(OCH(CH₃)CH₂)ₑOH, where e has an average value of 10;
(e) dioctyl ether;
(f) octylmethoxycinnamate and octyl salicylate;
(g) C12-C₁₈ alkyl benzoate and benzoate ester derivatives thereof selected from the group consisting of isostearyl benzoate and octyl dodecyl benzoate;
(h) isoparaffins having a distillation temperature in the range of 178-207 degrees C.;
(i) dioctyl carbonate;
(j) paraffins having a distillation temperature in the range of 372-426 degrees C.; and
(k) mixtures of any of the foregoing, provided that the clarity of the gelled shell is maintained.

One particular group of non-silicone organic materials is selected from the group consisting of C14-20 straight chain or branched fatty alcohols (especially isostearyl alcohol or octyldodecanol), isopropyl myristate, PPG-3 myristyl ether, octyl salicylate, isoparaffin with a flash point in the range of 60-130 degrees C., dioctyl ether, octyl methoxycinnamate, C12-15 alkyl benzoate.

Of course, the gelling agent must be soluble in the solvent system, at least at elevated temperatures, as described in U.S. Pat. No. 5,500,209.

The outer shell which is a gelled phase comprises a clear cosmetic composition which is a clear stick having a failure stress of at least 2 Pascals and which comprises:
(a) at least 7-15% (such as in the range of 10-15%) by weight based on the total weight of the outer phase of at least one siliconized polyamide of Formula IIIA: 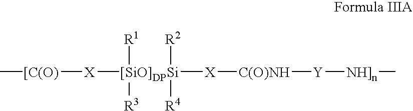
   where:
(1) DP (degree of polymerization) is a number in the range of 12-18;
(2) n is a number selected from the group consisting of 1-500 (particularly 20-200 and, more particularly 40-100 with an example being 40-130);
(3) X is a linear or branched chain alkylene having 1-30 carbons;
(4) Y is selected from the group consisting of linear and branched chain alkylenes having 1-40 carbons, wherein:
   (A) the alkylene group may optionally and additionally contain in the alkylene portion at least one of the members of a group consisting of (i) 1-3 amide linkages; (ii) C5 or C6 cycloalkane (as a cycloalkylene linkage); and (iii) phenylene optionally substituted by 1-3 members selected independently from the group consisting of C₁-C3 alkyls; and
   (B) the alkylene group itself may optionally be substituted by at least one member selected from the group consisting of (i) hydroxy; (ii) C3-C8 cycloalkane; (iii) 1-3 members selected independently from the group consisting of C1-C3 alkyls; phenyl optionally substituted by 1-3 members selected independently from the group consisting of C1-C3 alkyls; (iv) C1-C3 alkyl hydroxy; and (v) C1-C6 alkyl amine; or —Y—═—Z²⁻ where 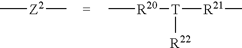
      wherein each of R²⁰, R²¹ are independently selected from the group consisting of linear and branched C1-C10 alkylenes; R²² is selected from the group consisting of linear and branched C1-C10 alkanes; and T is selected from the group consisting of (i) a trivalent atom selected from N, P and Al; and (ii)-CR, where R is selected from the group consisting of hydrogen, methyl, ethyl, propyl, isopropyl, a siloxane chain, and phenyl, wherein the phenyl may optionally be substituted by 1-3 members from the group consisting of methyl and ethyl, especially methyl and ethyl and most especially methyl; and
(5) each of R¹—R⁴ is independently selected from the group consisting of methyl, ethyl, propyl, isopropyl, a siloxane chain, and phenyl, wherein the phenyl may optionally be substituted by 1-3 members from the group consisting of methyl and ethyl (with more particular values for R¹—R⁴ being selected from methyl and ethyl and especially methyl);
   wherein the polyamide of Formula IIIA has:
   (i) a silicone portion in the acid side of the polyamide;
   (ii) an average molecular weight of at least 50,000 daltons (particularly in the range of 90,000-120,000) with at least 95% of the polyamide having a molecular weight greater than 10,000 daltons; and
   (iii) a polydispersity of less than 20 (particularly less than 10);

As noted above, the general class of polyamides from which further selections for the invention are made herein is the class generally described in copending case WO 99/06473.

The values for X, Y, DP, and R¹—R⁴ may be the same or different for each unit of the polyamide.

By siloxane groups is meant groups having siloxane units: 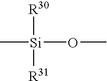
where R³⁰ and R³¹ are each independently selected from the group consisting of organic moieties, and each of R³⁰ and R³¹ are connected to the silicon by a carbon-silicon bond.

The carbon numbers in the alkylene chain do not include the carbons in the extra segments or substitutions. Also, the polyamides must have a siloxane portion in the backbone and optionally may have a siloxane portion in a pendant or branched portion.

If repeated with no variations in the defined variables, Formula IIIA is representative of a linear homopolymer. Acceptable variations of the invention include: (1) polyamides in which multiple values of DP, X, Y, and R¹—R⁴ occur in one polymeric molecule, wherein the sequencing of these units may be alternating, random or block; (2) polyamides in which an organic triamine or higher amine such as tris(2-aminoethyl)amine replaces the organic diamine in part, to produce a branched or crosslinked molecule; and (3) physical blends of any of (1) and (2) and/or linear homopolymers.

Particular examples of compounds of Formula IIIA include the following:
1) Polyamides of Formula IIIA where the values for X, Y, n, and DP are the same as defined in Formula IIIA (especially with DP=15), and R¹—R⁴ are each methyl;
2) Polyamides of Formula IIIA where the DP is in the range of 12-18 and the molecular weight is in the range of 90,000-120,000 daltons;
3) Polyamides of Formula IIIB: 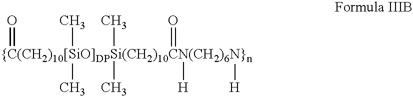
   where DP is from 12-18 and n has the same value as in Formula IIIA;
4) Polyamides of Formula IIIB wherein the DP is from 12-18 and the polyamide has a molecular weight in the range of 90,000-120,000 daltons;
5) Polyamides of Formula IIIB wherein the DP is 15;
6) Polyamides of Formula IIIB wherein the DP is 15 and the polyamide has a molecular weight in the range of 90,000-120,000 daltons;
7) Polyamides of Formula IIIA where the values of X, Y, DP and R¹—R⁴ remain the same in each unit of the polymer;
8) Polyamides of Formula IIIB where the value of DP and n remain the same for each unit of the polymer;
9) Polyamides of Formula IIIA containing multiple siloxane block lengths as shown in Formula IIIC: 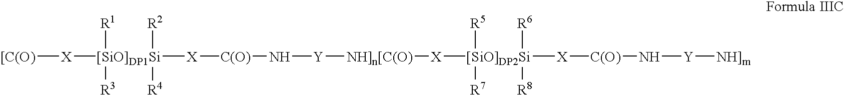
   where X, Y, n, and R¹—R⁴ have the meanings described above for Formula IIIA; m is selected from the same groups as defined for n, and n and m denote the total number of units enclosed within the brackets, with the individual units arranged with regular, alternating, block or random sequencing; R⁵—R⁸ is selected from the same group as defined for R¹—R⁴; DP1 and DP2 may be the same or different and are each independently selected from the same group as defined for DP; and the units denominated by n and m may be structured to form either block (regularly sequenced) or random copolymers.
10) Polyamides of Formula A containing siloxane block lengths of Formula IIIC wherein all of the R groups are selected to be methyl.
11) Polyamides of Formula IIIA containing siloxane block lengths of Formula IIIC wherein DP I=DP2.
12) Polyamides of Formula IIIA containing siloxane block lengths of Formula IIIC wherein all of the R groups are selected to be methyl and DP1=DP2.
13) Polyamides synthesized from multiple diamines as shown in Formula IIID: 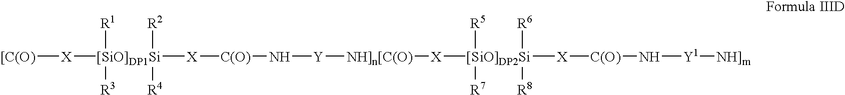
   where X, Y, m, n, and R¹—R⁸, DP1, DP2 have the same meanings as described above for Formula IIIA and Formula IIIC; Y¹ is independently selected from the same group as defined for Y; and the units denominated by n and m may be structured to form either block (regularly sequenced) or random copolymers.
14) Polyamides of Formula IIID where DP1=DP2.
15) Polyamides of Formula IIID where all of the R groups are selected to be methyl.
16) Polyamides of Formula IIID where all of the R groups are selected to be methyl and DP1=DP2.

In general, the siloxane-based polyamides (1) contain both siloxane groups and amide groups to thicken compositions containing silicone fluids (volatile and/or non-volatile silicone fluids); (2) are non-flowable solids at room temperature; and (3) dissolve in a fluid which contains silicone at a temperature of 25-160 degrees C. to form a translucent or clear solution at a temperature in this range.

With regard to the siloxane units in the siloxane-based polyamides, the siloxane units must be in the main or backbone chain but can also optionally be present in branched or pendent chains. In the main chain the siloxane units occur in segments as described above. In the branched or pendent chains the siloxane units can occur individually or in segments.

While the invention focuses on polyamides as described above with a DP=12-18, it is noted that some amount of polyamides of the same formulae but with a DP 5-30 may be included provided that clarity and structural integrity parameters are met.

Particular examples of such systems of siloxane-based polyamides include:
(a) polyamides of Formula IIIA where DP is a number in the range of 5-30, particularly 15-20, more particularly 12-18 and especially 15, provided that at least 7% of the composition is a polyamide of Formula IIIA with a DP in the range of 12-18, especially 15;
(b) physical blends of two or more polyamides described above in Formulae IIIA, IIIB, IIIC, IIID, IV and IVA, wherein (1) at least 80% of the blend is at least one polyamide as described above for this invention with a DP in the range of 5-30 with at least 7% of the final cosmetic composition being a polyamide of Formula IIIA with a DP in the range of 12-18, especially 15; and (2) the remainder of the blend is a polyamide of the Formulae IIIA, IIIB, IIIC, or IIID, except that the DP value is a number in the range of 45-500, or blends of these higher DP materials;
(c) compounds of Formula IIIC where (1) the value for DP₁=5-30 and the value for DP2=5-500 (more particularly, 5-100) and (2) the portion of the polyamide having DP I is about 1-99 weight % based on the weight of the total polyamide content and the portion of the polyamide having DP2 is about 1-99 weight % with at least 8% of the final cosmetic composition being a polyamide of Formula IIIC with a DP in the range of 12-18, especially 15;
(d) physical blends of polyamides of Formula IIIB made by combining (1) 60-99 weight % of a polyamide where DP=5-30 and especially where DP=10-20, and (2) 1-20 weight % of a polyamide where DP=5-500, especially where DP=45-100 with at least 8% of the final cosmetic composition being a polyamide of Formula IIIB with a DP in the range of 12-18, especially 15;
(e) polyamides of Formula IIID where at least one of Y and Y¹ contains at least one hydroxyl substitution with at least 8% of the final cosmetic composition being a polyamide of Formula IIID with a DP in the range of 12-18, especially 15;
(f) polyamides of Formula IIIA synthesized with at least a portion of an activated di-acid (diacid chloride, dianhydride or diester) instead of the diacid, with at least 7% of the final cosmetic composition being a polyamide of Formula IIIA with a DP in the range of 12-18, especially 15;
(g) polyamides of Formula IIIA where X═—(CH₂)₃— with at least 7% of the final cosmetic composition being a polyamide of Formula IIIA with a DP in the range of 12-18, especially 15;
(h) polyamides of Formula IIIA where X═—(CH₂)₁₀— with at least 7% of the final cosmetic composition being a polyamide of Formula IIIA with a DP in the range of 12-18, especially 15;
(i) polyamides of Formula IIIA where the polyamides are made with a monofunctional chain stopper selected from the group consisting of monofunctional amines, monofunctional acids, monofunctional alcohols, including fatty acids, fatty alcohols and fatty amines, such as, for example: octylamine, octanol, stearic acid and stearyl alcohol with at least 7% of the final cosmetic composition being a polyamide of Formula IIIA with a DP in the range of 12-18, especially 15.

Particular examples of (a)-(i) include those having a molecular weight of at least 50,000 daltons (particularly greater than 70,000 daltons, more particularly in the range of 80,000-150,000 (for example 80,000-90,000 daltons), and even more particularly in the range of 90,000-120,000 daltons) with at least 95% of the polyamide having a molecular weight greater than 10,000 as measured by size exclusion chromatography.

In general, when using polyamides of Formula IIIA to make antiperspirants and/or deodorants, an amount of polyamide equal to at least 7% by weight based on the final weight of the gelled shell. This is especially true if a polyamide of Formula IIIA having a DP=15 is used. If a polyamide with a DP=30 is used, about 5-15% more polyamide must be used to obtained the same resistance to stress.

For the outer shell, the polyamide gelling agent can be used in an amount of 7-80 percent by weight, more particularly 7-30%, and, even more particularly, 7-20% and, most particularly, 10-15 percent by weight based on the total weight of the outer phase. It is preferred that the primary gellant not exceed 50 percent by weight of the base composition of the outer phase. A co-gellant in the amount of 0-1 weight % based on weight of the outer shell can also be included as described below.

It should also be noted that nomenclature is being developed to call this type of polyamides “nylon/dimethicone copolymers” such as “nylon 611/dimethicone copolymer”, where “611” means that the organic portion of the copolymer has 6 and 11 carbons on either side of the amide group.

While one method for making polyamides is described in U.S. Patent Number U.S. Pat. No. 6,051,216 listed above, another method for making such polyamides is described in U.S. Pat. No. 5,981,680, both of which are incorporated by reference as to the methods of making such compositions.

Polyamides having a molecular weight in the range of 90,000-120,000 daltons and a degree of polymerization (DP) in the range of 12-18, especially 15, are especially useful in practicing the invention.

Optimizing the length of the siloxane portions of the molecule (the “DP”) involves a balancing of various considerations. Polyamides with long siloxane chains (for example, DP>50) tend to produce soft products in cyclomethicone. The efficiency of the gellant is improved by reducing the length of the siloxane units (that is, selecting and making a molecule with a DP<50), but the compatibility with cyclomethicone may be compromised as the DP decreases. For example, a polyamide synthesized from a siloxane diacid with a DP=15 and hexamethylene diamine does not produce clear products in cyclomethicone alone. However, transparent gels can be obtained if an organic emollient such as, for example, PPG-3 myristyl ether or isoparaffins is blended at various levels with the silicone fluids. As a result, polymers with DP=15 are preferred, so that the formulation for the resulting cosmetic composition has a combination of some compatibility with silicone fluids and good gelling efficiency. It should be noted that frequently more than one emollient is normally used to achieve the preferred aesthetics, and transparency, for example, with a DP=15.

In addition to the DP of the polyamide, the molecular weight must also, be considered. Polymers of extremely high molecular weight (for example, greater than 200,000 daltons) tend to produce rubbery, elastic gels and are less desirable. It has been found that optimal gellation occurs with polyamide gellants of molecular weight greater than 70,000 (particularly in the range of 90,000-120,000 daltons) as determined by size exclusion chromatography with universal calibration as described in Styring, J. E. et al “An Experimental Evaluation of a New Commercial Viscometric Detector for Size-Exclusion Chromatography (SEC) Using Linear and Branched Polymers,” J. Liquid Chromatograph, Volume 9, pages 783-804 (1986). In practicing the current invention, the optimal range of molecular weights for the primary gellant should be from 50,000-150,000 daltons, especially 70,000-120,000 daltons, and more especially 80,000-150,000 daltons, particularly 80,000-120,000 daltons (with a particular example being 80,000-90,000 daltons), and more particularly 90,000-120,000 daltons. It is believed, however, that incorporation of low levels of such high molecular weight species, for example, 0.5 weight % of a high molecular weight polyamide having a molecular weight in the range of 120,000-200,000 may give the base composition and cosmetic compositions made therefrom improved mechanical properties. Overall it is desired that at least 95% of the siliconized polyamide gellant have a molecular weight of at least 10,000 daltons.

It has been found that selecting siliconized polyamides with certain values for polydispersity and suitable stress/strain properties has an important affect on being able to form stick products. Polydispersity is calculated as M_{W}/M_{N} where M_{N} is number average molecular weight and M_{W} is weight average molecular weight. More particularly, when the molecular weight of the siliconized polyamide is increased while the polydispersity of the polyamide gellant remains narrow, the strength of the formulated product increases. The strength of the formulated product is monitored using a Three Point Bending technique as found in An Introduction to the Mechanics of Solids, (edited by Lardner, T. J.; McGraw-Hill 1978). A failure stress greater than 2.0 Pascals (and preferably greater than 4.0 Pascals) is desired for a stick product. If the failure stress is less than 2.0 Pascals, a softer stick can be formed.

As noted above, the siloxane-based polyamides used as thickening agents in base and cosmetic compositions of the present invention contain both siloxane units and amide linkages. The siloxane units provide compatibility with the silicone fluid (for example with the cyclomethicones), while the amide linkages and the spacing and selection of the locations of the amide linkages facilitate gellation and the formation of cosmetic products. While opaque as well as clear compositions may be formed, it is preferred that the cosmetic compositions formed be clear upon cooling a solution of a combination of the siloxane polyamide with the silicone and organic emollient component and leaves low to no white residue after application to the underarm area.

The siloxane-based polyamide gelling agent, can consist of one or more polyamides as described above (or a mixture of these polymers) as the sole gelling agent, or can contain the polyamide admixed with other thickening agents (including conventional gelling agents). The siloxane units provide compatibility with the silicone fluids. The amide portions are utilized reversibly for physically cross-linking purposes so as to form the gel.

Optionally, a co-gellant can be included. Particular examples of co-gellants include: 0-1 weight % dibenzylidene sorbitol (only in anhydrous systems); 0-1 weight % 12-hydroxy stearic acid; 0-1 weight % amine stearate; 0-1 weight % N,N′-hexamethylenebis-(10-undecenamide; 0-35 weight % silicone elastomer (for example, DC-9040 form Dow Corning Corp. or KSG-15 from Shin-Etsu); and especially 0-1 weight % (particularly 0.1-2.0 weight %) N-lauroyl glutamic acid amide (for example, GP-1 from Ajinomoto). In selecting the type and amount of co-gellant, attention to clarity must also be maintained. The amount of gellant+co-gellant should be maintained in the range of 7-20 weight % based on the weight of the entire stick product.

The transparent outer shell may also contain emollients of the type listed above for the core, provided attention is paid to maintaining clarity of the shell. Particular examples of emollients of interest for the outer shell are selected from the group consisting of non-silicone emollients which are either themselves or in mixtures with other solvents miscible in the originally selected silicone fluid (for example, C14-C20 straight or branched chain fatty alcohols (especially isostearyl alcohol and octyldodecanol)), isopropyl myristate, PPG-3 myristyl ether, octyl salicylate, isoparaffins, dioctyl ether, PPG-10 cetyl ether, octyl methoxycinnamate), and C12-15 alkyl benzoate (for example, FINSOLV TN from Finetex Inc., Elmwood Park, N.J.). Another group of such emollients comprises guerbet alcohols (such as isocetyl alcohol or isostearyl alcohol); esters having 14-22 carbons (such as isopropyl palmitate, isopropyl isostearate, octyl stearate, hexyl laurate and isostearyl lactate); and a liquid mixture of hydrocarbons which are liquids at ambient temperatures (such as petroleum distillates and light mineral oils). The emollients used in the shell can be included in the compositions of the present invention in amounts within the range of 0-70%, preferably 5-25%, by weight, of the total weight of the composition.

Other parameters may also be considered. These are:
(1) degree of impurities in the siliconized polyamide;
(2) use of a particular combination of silicone, organosilicone and organic emollients; and
(3) the concentration of gellant in the formulation.

The level of impurities in the gelling agent (impurities being defined as any material having a molecular weight below 4000 daltons as measured by size exclusion chromatography) should be kept low, particularly less than 5% by weight, based on the weight of the polyamide.

Optionally a surfactant may be included in the outer shell. Surfactants illustratively include alkanolamides (such as N-alkyl pyrrolidone), ethoxylated amides (for example, the polyethylene glycol amide of tallow acid that conforms generally to the formula RC(O)—NH—(CH₂CH₂O)ₙH where RCO— represents the fatty acids derived from tallow and n has an average value of 50 (also called PEG-50 tallow amide)); amine oxides (for example, cocamidopropylamine oxide); ethoxylated carboxylic acids (for example, the polyethylene glycol diester of lauric acid that conforms generally to the formula CH₃(CH₂)₁₀C(O)—(OCH₂CH₂)ₙO—C(O)(CH₂)₁₀CH₃ where n has an average value of 8 (also called PEG-8 dilaurate)); ethoxylated glycerides (for example, a polyethylene glycol derivative of Castor Oil with an average of 4 moles of ethylene oxide (also called PEG-4 castor oil)); glycol esters (for example, propylene glycol ricinoleate); monoglycerides (for example, glycerol myristate); polyglyceryl esters (for example, polyglyceryl-4 oleyl ether); polyhydric alcohol esters and ethers (for example, sucrose distearate); sorbitan/sorbitan esters (for example, sorbitan sesquiisostearate); triesters of phosphoric acid (for example, trioleth-8 phosphate (a material which is predominantly the triester of phosphoric acid and ethoxylated oleyl alcohol with an average of 8 moles of ethylene oxide)); ethoxylated alcohols (for example, laureth-4); ethoxylated lanolin (for example, a polyethylene glycol derivative of Lanolin with an average of 20 moles of ethylene oxide (also called PEG-20 lanolin)); ethoxylated polysiloxanes (for example, dimethicone copolyol); propoxylated polyoxyethylene ethers (for example, the polyoxypropylene, polyoxyethylene ether of cetyl alcohol that conforms generally to the formula CH₃(CH₂)₁₄CH₂(OCH(CH₃)CH₂)ₓ(OCH₂CH₂)_{y}OH where x has an average value of 5 and y has an average value of 20 (also called PPG-5 ceteth-20)); and alkylpolyglycosides (for example, lauryl glucose). The surfactant (or surfactant blend) includes non-ionic compounds, and can also include blends thereof with cationic (for example, the polyethylene glycol amine of tallow acid that conforms generally to the formula R—NH—(CH₂CH₂O)ₙH (also called PEG-15 tallow amine)) or anionic (for example, sodium lauroyl isethionate which is the sodium salt of the lauric acid ester of isethionic acid) surfactants.

The surfactant or blend thereof incorporated in compositions according to the present invention can, illustratively, be included in amounts of 0-3% by weight (for example, at least 0.5 weight %), of the total weight of the composition.

The formulations of the outer shell of this invention will be made without antiperspirant salt.

In contrast to co-pending case U.S. Ser. No. 9/922,091 (2002/0048557) which describes opaque to translucent products, the products of this invention have a clear shell and an opaque core and yet still provide low to no white residue.

The firmness or hardness of the individual phases (clear outer phase and opaque inner core) will be about 600 gram-force to about 5,000 gram-force, preferably from about 750-2,000 gram-force, and more preferably from about 800-1400 gram-force as described by the following test. The term “hardness” as used herein is based on how much force is required to move a penetration cone a specified distance and at a controlled rate into the opaque or clear part of the stick product of the invention. Higher values represent harder product, and lower values represent softer product. These values are measured at 27 degrees C., 15% relative humidity, using a TA-XT2 texture Analyzer, from Texture Technology Corp., Scarsdale, N.Y. The product hardness value as used herein represents the peak force required to move a standard 45 degree angle penetration cone through the composition for a distance of 10 mm at a rate of 2 mm/sec. The standard cone is also available from Texture Technology Corp., as a part number TA-15, and has a total cone length of about 24.7 mm, angled cone length of about 18.3 mm, a maximum diameter of the angled surface of the cone of about 15.5 mm. The cone is smooth, stainless steel construction and weighs about 17.8 grams.

The outer phase may also contain other optional ingredients such as fragrance, coloring agents, antibacterials (antimicrobials) and other conventional ingredients known to those in the art for formulating such products to form cosmetic compositions.

Throughout the present specification, “antiperspirant active” and “deodorant active” materials are discussed. Both types of materials contribute to reduction of body malodor, for example, axillary malodor. By reduction of body malodor, it is meant that, generally, there is less body malodor after application of the composition to a person's skin, as compared to a person's malodor without application of the composition. Such reduction can be due to a masking of the malodor, absorption and/or chemical reaction of the malodorous material, reduction of the levels of the bacteria producing the malodorous materials, for example, from perspiration, reduction of perspiration, etc. The antiperspirant active materials, when utilized in appropriate amounts, primarily act to reduce malodor by reducing perspiration; the antiperspirant active materials can also have a deodorant function, for example, as an antimicrobial or bacteriostatic agent. The deodorant active materials do not substantially reduce perspiration, but reduce malodor in other ways. For example, as fragrances masking the malodor or reducing the malodor intensity; absorbents; antimicrobial (bacteriostatic) agents; or agents chemically reacting with malodorous materials.

Where deodorant active materials are incorporated in compositions according to the present invention, so as to provide deodorant compositions, conventional deodorant fragrances and/or antimicrobial agents can be incorporated as the deodorant active materials. A fragrance would, illustratively, be incorporated in an amount of 0.5%-3.0% by weight, of the total weight of the composition. Known bacteriostats include bacteriostatic quaternary ammonium compounds such as 2-amino-2-methyl-1-propanol (AMP), cetyl-trimethylammonium bromide, cetyl pyridinium chloride, 2, 4, 4′-trichloro-2′-hydroxydiphenylether (Triclosan), N-(4-chlorophenyl)-N′-(3,4-dichlorophenyl)urea (Triclocarban) and various zinc salts (for example, zinc ricinoleate). The bacteriostat can, illustratively, be included in the composition in an amount of 0.1-1.0% by weight, of the total weight of the composition. Triclosan, can illustratively be included in an amount of from 0.1% to about 0.5% by weight, of the total weight of the composition.

As indicated previously, the compositions according to the present invention are sticks with varying degrees of rigidity depending on amounts of thickening agent incorporated in the composition.

Base and cosmetic compositions according to the present invention can easily be manufactured by methods known to those skilled in the art such as by using known mixing procedures. Base compositions according to the present invention can be made by mixing the various components at an elevated temperature (that is, by heating and mixing the various components) and then cooling in order to form the gelled (solidified) stick composition. For cosmetic compositions, the additional ingredients are added using techniques and at times in the manufacturing process as are known to those in the art. Desirably, any volatile components (such as fragrances) are added to the mixture at a relatively late stage of the mixing, so as to limit volatilization of the volatile components.

In making the sticks of this invention the opaque core is prepared in its desired geometric configuration by using a suitable compressor as described in the Examples such as a Carver die compressor (from Carver Inc., Wabash, Ind.).

Stick compositions of the present invention can be applied by elevating the stick out of the package so as to expose the end of the stick, and then rubbing the end of the stick on the skin in order to deposit stick material (including antiperspirant active) on the skin. Thus, the active material on the skin is available to reduce body malodor and/or reduce the flow of perspiration from, for example, the axillary regions of the body.

Throughout the present specification, where compositions are described as including or comprising specific components or materials, or where methods are described as including or comprising specific steps, it is contemplated by the inventors that the compositions of the present invention also consist essentially of, or consist of, the recited components or materials, and also consist essentially of, or consist of, the recited steps. Accordingly, throughout the present disclosure any described composition of the present invention can consist essentially of, or consist of, the recited components or materials, and any described method of the present invention can consist essentially of, or consist of, the recited steps.

Throughout the specification and claims all percents are in percents by weight unless stated otherwise. If no standard is indicated, then the percent by weight is in reference the total weight of the cosmetic composition.

A desired feature of the present invention is that a clear, or transparent, outer phase is provided. The term clear or transparent according to the present invention is intended to connote its usual dictionary definition; thus, a clear, for example, stick or gel antiperspirant composition of the present invention allows ready viewing of objects behind it. By contrast, a translucent composition, although allowing light to pass through, causes the light to be scattered so that it will be impossible to see clearly objects behind the translucent composition. An opaque composition does not allow light to pass therethrough. Within the context of the present invention, a gel or stick is deemed to be transparent or clear if the maximum transmittance of light of any wavelength in the range 400-800 nm through a sample 1 cm thick is at least 35%, preferably at least 50%. The gel or stick is deemed translucent if the maximum transmittance of such light through the sample is between 2% and less than 35%. A gel or stick is deemed opaque if the maximum transmittance of light is less than 2%. The transmittance can be measured by placing a sample of the aforementioned thickness into a light beam of a spectrophotometer whose working range includes the visible spectrum, such as a Bausch & Lomb Spectronic 88 Spectro-photometer. As to this definition of clear, see European Patent Application Publication No. 291,334 A2. Thus, according to the present invention, there are differences between transparent (clear), translucent and opaque compositions.

Products of varying clarity and transparency can be formed. Clear cosmetic compositions can be formed if all the components of the particular cosmetic composition are soluble in each other, resulting in a single phase product. Clear cosmetic compositions can also be prepared from multiple phase compositions, for example, an emulsion or suspension, if each phase individually is clear and the refractive index of each phase is matched. Additionally, clear cosmetic compositions can be made from multiple phase compositions if the droplet (particle) size of the internal phase(s) are small enough, less than 0.2 micron. Examples of this are microemulsions and very fine particles in suspension. If the aforementioned conditions are not met, the cosmetic compositions will exhibit various degrees of transparency and opacity.

In the following, specific synthesis examples for forming siloxane-based polyamides of this invention are set forth, and specific examples of antiperspirant and deodorant compositions within the scope of the present invention are also set forth. These specific synthesis examples and examples are illustrative in connection with the present invention, and are not limiting. In the following, as well as throughout the present disclosure, names utilized are the CTFA (Cosmetics, Toiletry and Fragrance Association, Inc.) names, as set forth in the CTFA International Cosmetic Ingredient Dictionary (4th Ed. 1991), the contents of which dictionary are incorporated herein by reference in their entirety. Throughout the description of this invention chemical abbreviations and symbols have their usual and customary meanings, temperatures are in degrees C., all percents are in weight percents based on the total weight of the composition, and comprising shall be interpreted as including as subgroups consisting of and consisting essentially of. While particular siloxane-based polyamides are disclosed or used in the following Examples, it is to be understood that other siloxane-based polyamides meeting the criteria of the invention may also be substituted for the particular siliconized polyamide used in the examples and such compositions are within the spirit and scope of the invention.

### EXAMPLES

### Example 1

A core for the product of the invention may be made with the desired geometric configuration by combining 25 weight % powdered aluminum zirconium trichlorohydrex-glycine (AZP908 from Reheis, Berkeley Heights, N.J.); 12.5 weight % talc (Ultr Chem, Whittaker Clark & Daniels, South Plainfield, N.J.) and 5 weight % dimethicone copolyol (DC193, Dow Corning, Midland Mich.). The ingredients were combined and compressed. The opaque core is made by mixing the ingredients in a V-shell mixer for about 30 minutes. The mixed powder is added to a die with the desired geometric configuration. The die containing the powder is then placed between two metal plates of a suitable compressor such as the CARVER, manufactured by Carver, Inc. The pressure used to compress the powder is between 2.27-227 kilograms (5-500 pounds). After compression is completed, the core is pushed out using appropriate tools.

### Example 2

The gelled shell portion of the formula is made separately by or melting the gellant along with the silicone and non-silicone organic materials (collectively referred to as the solvent) at about 90 degrees C. A composition for the shell is made by combining together 20.7 weight % PPG-3 myristyl ether, 25.9 weight % cyclomethicone (DC 245 from Dow Corning), 5.7 weight % Nylon 611/dimethicone copolymer with a DP=15, Dow Corning), 4 weight % isostearyl alcohol, 0.6 weight % dibutyl lauroyl glutamide, and 0.6 weight % fragrance with heating.

### Example 3A

A complete product is made by positioning the core from Example 1 in an upright container such as an oval shape or cylindrical shape (concentric cylinders are preferred) and then pouring the composition of Example 2 in melted form (about 75 degrees C.) around the core. The product is then allowed to cool at room temperature and solidify.

### Example 3B

The mixture is then poured into a barrel of the type typically used for antiperspirant/deodorant products (ovoid shape). The core portion of the product is inserted into the gelled shell portion of the product (usually in a centered position) prior to the solidification of the gelled shell by cooling. After cooling is completed to room temperature, the final product is ready for use.

### Example 4

The method of Example 1 may be repeated with the following ingredients: 18 weight % aluminum zirconium tetrachlorohydrex-glycine (AZP 908, from Reheis, Berkeley Heights, N.J.); 8 weight % zinc oxide, 8.5 weight % beeswax synthetic beeswax from Koster Keunen Inc., Watertown, Conn.); 5 weight % polyacrylic acid (Acusol 15104R from Rohm & Haas, Philadelphia, Pa.).

### Example 5

The method of Example 2 may be repeated with the following ingredients: 13 weight % PPG-3 myristyl ether (Varonic APM, from Goldschmidt Chem, Essen, Germany), 22 weight % cyclomethicone (DC 245, Dow Corning, Midland, Mich.), 5 weight % Nylon 611/dimethicone copolymer with a DP=15, Dow Corning, Midland, Mich.), 4 weight % isostearyl alcohol, 0.5 weight % dibutyl lauroyl glutamide (GP-1, Ajinomoto U.S., Paramus, N.J.), and 0.5 weight % fragrance.

### Example 6

A complete product is made by positioning the core from Example 4 in an upright container such as an oval shape or cylindrical shape (concentric cylinders are preferred) and then pouring the composition of Example 5 in melted form (about 75 degrees C.) around the core. The product is then allowed to cool at room temperature and solidify.

### Examples 7 and 8

Each of Examples 1, 2, 4 and 5 may be repeated with minor amounts (0.1-3 weight %) of one or more of dyes, pigments, additional fragrance (for example, adding encapsulated fragrance to the core, adding pigments to the core, adding colored dyes to the shell while maintaining clarity of the shell).

1. A dual phase stick antiperspirant and/or deodorant product comprising an opaque non-gelled inner core as 20-70 weight % of the total composition and a clear outer shell as 30-80 weight % of the total composition wherein:
(a) based on the weight of the inner core as 100%, the composition of the inner core comprises:
(i) 20-70 weight % of an antiperspirant active powder, provided that the total amount of antiperspirant active in the total product does not exceed 25 weight %;
(ii) 0.1-20 weight % of a filler;
(iii) 2-20 weight % of a binder;
(iv) 1-20 weight % of an emollient;
(v) optionally 0-3 weight % of a surfactant; and
(vi) optionally 0-3 weight % of one or more members selected from the group consisting of coloring agents, and fragrances;
(b) based on the weight of the outer shell as 100%, the clear outer shell is positioned around the inner core and comprises:
(i) 7-15 weight % of siliconized polyamide Formula IIIA: 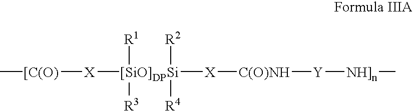
where:
(1) DP is a number in the range of 12-18;
(2) n is a number selected from the group consisting of 1-500;
(3) X is a linear or branched chain alkylene having 1-30 carbons;
(4) Y is selected from the group consisting of linear and branched chain alkylenes having 1-40 carbons, wherein:
(A) the alkylene group may optionally and additionally contain in the alkylene portion at least one of the members of a group consisting of (i) 1-3 amide linkages; (ii) C5 or C6 cycloalkane as a cycloalkylene linkage; and (iii) phenylene optionally substituted by 1-3 members selected independently from the group consisting of C1-C3 alkyls; and
(B) the alkylene group itself may optionally be substituted by at least one member selected from the group consisting of (i) hydroxy; (ii) C3-C8 cycloalkane; (iii) 1-3 members selected independently from the group consisting of C1-C3 alkyls; phenyl optionally substituted by 1-3 members selected independently from the group consisting of C1-C3 alkyls; (iv) C1-C3 alkyl hydroxy; and (v) C1-C6 alkyl amine;
or —Y—═—Z²⁻ where 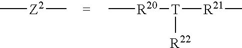
wherein each of R²⁰, R²¹ are independently selected from the group consisting of linear and branched C1-C10 alkylenes; R²² is selected from the group consisting of linear and branched C1-C10 alkanes; and T is selected from the group consisting of (i) a trivalent atom selected from N, P and Al; and (ii)-CR, where R is selected from the group consisting of hydrogen, methyl, ethyl, propyl, isopropyl, a siloxane chain, and phenyl, wherein the phenyl may optionally be substituted by 1-3 members from the group consisting of methyl and ethyl; and
(5) each of R¹—R⁴ is independently selected from the group consisting of methyl, ethyl, propyl, isopropyl, a siloxane chain, and phenyl, wherein the phenyl may optionally be substituted by 1-3 members from the group consisting of methyl and ethyl;
wherein the polyamide of Formula IIIA has:
(A-1) a silicone portion in the acid side of the polyamide;
(A-2) an average molecular weight of at least 50,000 daltons with at least 95% of the polyamide having a molecular weight greater than 10,000 daltons; and
(A-3) a polydispersity of less than 20;
(ii) 8-80 weight % of a silicone component comprising at least 8-30 weight % of a volatile silicone and optionally 0-30 weight % of a non-volatile silicone;
(iii) 10-70 weight % of a non-silicone organic material;
(iv) optionally a co-gellant selected from the group consisting of: 0-1.7% dibenzylidene sorbitol (for anhydrous systems); 0-0.5% 12-hydroxy stearic acid; 0-0.5% amine stearate; 0-0.5% N,N′-hexamethylenebis-(10-undecenamide; 0-5.0% silicone elastomer; and 0-1% N-lauroyl glutamic acid amide;
(v) optionally 0-5 weight % of a glycol component; and
(vi) 0-3 weight % of a surfactant;
wherein the outer gelled phase comprises the polyamide in combination with the silicone and non-silicone organic materials so that the ratio of the non-silicone organic materials to the organosilicone materials is in the range of 10:1-0.01:1.

2. A stick product according to claim 1 wherein n=20-200.

3. A stick product according to claim 1 wherein n=40-130.

4. A stick product according to claim 1 wherein n=40-100.

5. A stick product according to claim 1 wherein the molecular weight of the polyamide of Formula IIIA is in the range of about 90,000-120,000.

6. A stick product according to claim 1 wherein the polydispersity of the polyamide of Formula IIIA is less than 10.

7. A stick product according to claim 1 wherein the filler is selected from the group consisting of (a) inorganic oxides selected from the group consisting of aluminum oxide, titanium dioxide and zinc oxide; (b) silicates selected from the group consisting of silica, fumed silica and magnesium silicates; (c) clay selected from the group consisting of inorganic clays, kaolin and bentonite; (d) particulates selected from the group consisting of talc, talcum powder, zinc stearate, cornstarch, polyethylene in the size range of 10-50 microns, Nylon-12; and (e) mixtures of any of the foregoing.

8. A stick product according to claim 1 wherein the binder is selected from the group consisting of (a) acrylates copolymers selected from the group consisting of acrylates/polyvinylpyrrolidone copolymers and acrylates/vinyl acrylate copolymers; (b) acrylic acid and its copolymers selected from the group consisting of acrylic acid/acrylonitrogens copolymers; (c) polyvinylpyrrolidone; (d) copolymers of polyvinylpyrrolidone selected from the group consisting of polyvinylpyrrolidone/vinyl acrylate copolymers); (e) agar; (f) algin; (g) C10-200 alkyl alcohols; (h) waxes having a melting point in the range of 40-100 degrees C.; (i) gums selected from the group consisting of cellulose gums and xanthan gums; (j) celluloses selected from the group consisting of hydroxyethylcellulose and hydroxypropylcellulose; (k) gelatin; (l) starches selected from the group consisting of glyceryl starch, cornstarch; (m) lanolins; (n) guars selected from the group consisting of hydroxypropyl guar and carboxymethyl hydroxypropyl guar); (o) isopropyl/isostearyl esters; (p) polyethylene glycols selected from the group consisting of PEG-32, PEG-350, and PEG-90M; (q) an agent selected from the group consisting of dibenzylidene sorbitol, 12-hydroxystearic acid, and N-acyl amino acids; (r) dimethicone copolyol; and (s) mixtures of any of the foregoing.

9. A stick product according to claim 1 wherein the binder is selected from the group consisting of C10-30 alkyl alcohols.

10. A stick product according to claim 1 wherein the binder is selected from the group consisting of behenyl alcohol, lanolin alcohol, and polyvinyl alcohol having 50-200 carbons.

11. A stick product according to claim 1 wherein the binder is selected from the group consisting of natural, synthetic or modified waxes selected from the group consisting of beeswax, candelilla wax, synthetic wax, and silicone wax.

12. A stick product according to claim 1 comprising 1-10 weight % of the emollient.

13. A stick product according to claim 1 wherein the emollient is selected from the group consisting of:
(a) ethoxylated/propoxylated surfactants having 4-48 carbon atoms and 3-15 EO or PO units;
(b) oils selected from the group consisting of mineral oil, avocado oil, castor oil, corn oil, jojoba oil, safflower oil, sesame oil, soybean oil, and squalene;
(c) hydrogenated oils selected from the group consisting of hydrogenated vegetable oil, hydrogenated jojoba oil, and hydrogenated castor oil;
(d) volatile silicones; and
(f) mixtures of any of the foregoing.

14. A stick product according to claim 1 wherein the emollient is selected from the group consisting of:
(a) ethoxylated/propoxylated surfactants having 4-48 carbon atoms and 3-15 ethylene oxide or propylene oxide units;
(b) oils selected from the group consisting of mineral oil, avocado oil, castor oil, corn oil, jojoba oil, safflower oil, sesame oil, soybean oil, and squalene;
(c) hydrogenated oils selected from the group consisting of hydrogenated vegetable oil, hydrogenated jojoba oil, and hydrogenated castor oil;
(d) and mixtures of any of the foregoing.

15. A stick product according to claim 1 wherein the emollient is a non-silicone organic material selected from the group consisting of:
(a) esters selected from the group consisting of isopropyl myristate and C12-15 alkyl lactate;
(b) guerbet alcohols having 8-30 carbons;
(c) fatty alcohols selected from the group consisting of stearyl alcohol, myristyl alcohol, oleyl alcohol, and isocetyl alcohol;
(d) ethoxylated and propoxylated alcohols selected from the group consisting of polyethylene glycol ether of lauryl alcohol that conforms to the formula CH₃(CH₂)₁₀CH₂(OCH₂CH₂)_{b} OH where b has an average value of 4; PPG-14 butyl ether, where the PPG-14 portion is a polymer of propylene oxide that conforms to the formula H(OCH₂C(CH₃)H)ₙOH, where c has an average value of 14; PPG-3 myristyl ether which is the polypropylene glycol ether of myristyl alcohol that conforms to the formula CH₃(CH₂)₁₂CH₂(OCH(CH₃)CH₂)_{d}OH where d has an average value of 3; and PPG-10 cetyl ether which conforms to the formula CH₃(CH₂)₁₄CH₂(OCH(CH₃)CH₂)ₑOH, where e has an average value of 10;
(e) dioctyl ether;
(f) octylmethoxycinnamate and octyl salicylate;
(g) C12-C18 alkyl benzoate and benzoate ester derivatives thereof selected from the group consisting of isostearyl benzoate and octyl dodecyl benzoate;
(h) isoparaffins having a distillation temperature in the range of 178-207 degrees C.;
(i) dioctyl carbonate;
(j) paraffins having a distillation temperature in the range of 372-426 degrees C.; and
(k) mixtures of any of the foregoing, provided that the clarity of the gelled shell is maintained.

16. A stick product according to claim 15 wherein the non-silicone organic material is selected from the group consisting of isolauryl alcohol, isocetyl alcohol, and isostearyl alcohol.

17. A stick product according to claim 15 wherein the non-silicone organic material is selected from the group consisting of C12-C15 alkyl benzoate, isostearyl benzoate and octyl dodecyl benzoate.

18. A stick product according to claim 15 wherein the non-silicone organic material is selected from the group consisting of C14-20 straight chain or branched fatty alcohols, isopropyl myristate, PPG-3 myristyl ether, octyl salicylate, isoparaffin with a flash point in the range of 60-130 degrees C., dioctyl ether, octyl methoxycinnamate, and C12-15 alkyl benzoate.

19. A stick product according to claim 1 wherein the volatile silicone is a D5 cyclomethicone, a D6 cyclomethicone or mixture of D5 and D6 cyclomethicones.

20. A stick product according to claim 1 wherein the surfactant is polysorbate 20 or polysorbate 80.

21. A stick product according to any one of claims 1-20 which is free of any silicones in the inner core.
